(19) United States
(12) Patent Application Publication
(10) Pub. No.: US 2013/0041283 A1
(43) Pub. Date: Feb. 14, 2013

(54) Constraint Modified Signal

(71) Applicant: Brian D. Wichner, Otter Rock, OR (US)

(72) Inventor: Brian D. Wichner, Otter Rock, OR (US)

(21) Appl. No.: 13/655,441

(22) Filed: Oct. 18, 2012

(51) Int. Cl. A61B 5/053; A61N 1/36

(52) U.S. Cl. 600/547; 607/62

(57) ABSTRACT

Subject matter includes a method comprising: applying a first electrical signal to a subject; identifying a proposal to change the first electrical signal to a proposed second electrical signal; calculating at least one parameter of the proposed second electrical signal; and determining whether the calculated at least one parameter violates any rules. If the at least one parameter does violate any of the rules, then the method further comprises iteratively modifying one or more features of the proposed second electrical signal to arrive at a modified second electrical signal that complies with the rules. If the at least one parameter does not violate any of the rules, then the method further comprises applying the proposed second electrical signal to the subject.

### BACKGROUND

1. Field

Subject matter disclosed herein relates to modifying a waveshape of an electric signal to any configuration while being constrained to comply with various parameters, such as those set forth by a regulatory agency.

2. Information

A number of techniques for treating a subject (e.g., a patient) or detecting a physical condition of a subject may involve applying electrical energy via electrodes in contact with the subject. Such electrodes may comprise pads having an adhesive (or a water-activated adhesive) to temporarily affix the pads to a portion of a subject. For example, a transcutaneous electrical nerve stimulation (TENS) device may apply electric current to a subject via electrode pads to stimulate nerves of the subject for therapeutic purposes. In another example, muscle loss of a subject may be determined using electric impedance myography (EIM), which may measure resistance of a muscle to an electrical current by passing an amount of current through the muscle using two electrodes.

Governmental regulatory entities, among others, may establish rules that limit various parameter values of electrical signals applied to a subject. Accordingly, it may be undesirable to exceed particular thresholds that would violate such rules.

### BRIEF DESCRIPTION OF THE FIGURES

Non-limiting and non-exhaustive embodiments will be described with reference to the following figures, wherein like reference numerals refer to like parts throughout the various figures unless otherwise specified.

FIG. 1 is a cross-sectional schematic diagram illustrating electrodes for applying one or more electrical signals to a portion of a subject, according to an embodiment.

FIG. 2 is a schematic diagram illustrating at device displaying a signal that may be applied to a subject, according to an embodiment.

FIGS. 3-6 show example signal waves plotted as magnitude of voltage or current versus time, according to embodiments.

FIG. 7 shows a first signal wave modified to a second signal wave, plotted as magnitude of voltage or current versus time, according to an embodiment.

FIGS. 8A and 8B shows a first signal wave and a second signal wave, respectively, plotted as magnitude of voltage or current versus time, according to an embodiment.

FIGS. 9 and 10 show example signal waves plotted as magnitude of voltage or current versus time, according to embodiments.

FIG. 11 is a flow diagram of a process for determining whether to modify a signal, according to an embodiment.

FIG. 12 is a flow diagram of a process for modifying a proposed signal, according to an embodiment.

FIG. 13 shows a first signal wave in a process of being modified to a second signal wave, plotted as magnitude of voltage or current versus time, according to an embodiment.

FIG. 14 is a schematic block diagram illustrating a system for applying a signal to a subject, according to an embodiment.

FIG. 15 is a schematic diagram illustrating an embodiment of a computing system including a memory module.

### DETAILED DESCRIPTION

Reference throughout this specification to “one embodiment” or “an embodiment” means that a particular feature, structure, or characteristic described in connection with the embodiment is included in at least one embodiment of claimed subject matter. Thus, the appearances of the phrase “in one embodiment” or “an embodiment” in various places throughout this specification are not necessarily all referring to the same embodiment. Furthermore, the particular features, structures, or characteristics may be combined in one or more embodiments.

Impedance may refer to the opposition that a path of electrical current presents to the passage of the current if a voltage is applied. For example, in quantitative terms, impedance may comprise a complex ratio of the voltage to the current. Impedance (e.g., for time-varying electrical signals) may comprise an extension of the concept of resistance (e.g., non-time-varying electrical signals), and may include both magnitude and phase, unlike resistance, which may only include magnitude. In situations involving time-varying electrical signals, mechanisms in addition to normal resistance (e.g., ohmic resistance for non-time-varying electrical signals) may impede flow of current. Such mechanisms may comprise induction of voltages in conductors self-induced by magnetic fields of currents (inductance), and electrostatic storage of charge induced by voltages between conductors (capacitance). Impedance based, at least in part, on these two effects may collectively be referred to as reactance and forms an imaginary part of complex impedance whereas resistance forms a real part, for example.

The terms “resistance” and “impedance” are used herein interchangeably to mean the same thing unless used in the context of a sentence that indicates otherwise. For example, “resistance” means impedance that may comprise an inductive reactance, capacitive reactance, and/or ohmic resistance. On the other hand, “impedance” may mean ohmic resistance and may or may not include inductive reactance and/or capacitive reactance. Again, a context or description of a sentence or portion of text in which such terms are used may indicate one meaning over another meaning. The term “resistance” may comprise inductive reactance, capacitive reactance, and/or ohmic resistance. If “resistance” is intended to exclude inductive reactance and/or capacitive reactance then the term “ohmic resistance” is used.

The term “subject” is recited in examples herein. Unless otherwise described, a subject may comprise human, animal, fish, reptile, bird, and so on. A subject may also comprise abiotic systems or material, such as liquid, mineral, plastics, etc., although example embodiments are directed to biotic systems. For example, embodiments of techniques described may be applied in cases where a subject is human or where a subject is a fish or animal, and claimed subject matter is not limited in this respect. To describe a particular implementation, techniques may be applied to diagnose a physical condition (e.g., muscle mass, cancer, blood chemistry, and so on) of a human subject. In another implementation, techniques may be applied to perform research regarding any of a number of physical parameters of various aquatic species. In the latter implementation, the “subject” may comprise a particular aquatic specimen. Other implementation may involve animals, and so on. Accordingly, though the following descriptions may indicate a human subject, claimed subject matter is not limited in this respect.

Biological elements of a subject may comprise any portion or combination of portions of the subject, such as skin, muscle tissue, organs, normal or cancer cells, blood, ligaments, tendons, bones, scar tissue, and so on. Such biological elements may be microscopic or macroscopic. Such biological elements may be in any type of condition, such as healthy or normal, damaged or injured, deteriorated, inflamed, and so on.

In some embodiments, applications of electrical energy (e.g. for muscle stimulation, physical diagnosis, and so on) may involve a power source, a signal generator, at least two electrodes, and leads (e.g., cables, wires, conductors, and so on). Electrical energy application may comprise transcutaneous application, involving leads or electrodes on skin of a subject, for example.

An electrical signal may have properties that satisfy particular criteria. For example, energy per pulse of a signal applied to a patient may be below a particular upper limit set forth by a regulatory agency. Modifying such an electrical signal, however, may give rise to altered properties that no longer satisfy particular criteria. For example, increasing a pulse width of a signal may increase energy per pulse beyond a particular upper limit set forth by a regulatory agency. In some implementations, the waveshape of a signal may be graphically modified using any of a mouse, touchscreen, keyboard, and so on. For example, a user may use a mouse to graphically drag a portion (e.g., a point or a range of points) of a wave of a signal to modify the waveshape. A user may be unable to know how to modify a waveshape so as to maintain signals properties that satisfy particular criteria. Herein, “criteria” is used interchangeably with “criterion”, so that “criteria” means a single criterion or multiple criteria. Also, unless the context of a sentence indicates otherwise, “criteria” may be used interchangeably with the term “rules”, which is intended to comprise plural or singular.

Criteria or rules set forth by an agency or other entity need not be “hard-wired” into electronics or software code of a device. For example, a regulatory agency may set forth a rule that a signal applied to a patient is not to exceed a peak voltage of 35.0 volts. A device may be constructed with electronic components or software so that the device does not have a capability to exceed a peak voltage of 35.0 volts. However, in some embodiments, a device may be “over-built” in a sense that the device may be able to exceed limitations or operate beyond ranges set forth by an agency, group, or individual (e.g., a device may be built to exceed a peak voltage of 35.0 volts). Output capability, instead, may be reigned in or limited by values maintained in a memory. Such values may be updated from time to time or periodically. Such values may comprise data in a look-up table, for example. Values may comprise any of a number of parameters that describe an electronic signal, such as voltage, current, energy per pulse, power, frequency, rate of change of voltage, waveshape (e.g., ramp, sinusoid, square, or arbitrary shape), and so on. Thus, returning to the example, above, a device may be constructed to reach peak voltages of 100 volts, and include memory storing a variable that specifies output of the device is to not exceed a peak voltage of 35.0 volts. Such a device may provide a number of advantageous, including a device that may easily be adaptable to changing regulations, conditions, or preferences, for example.

In some embodiments, a device may be constructed so as to generate an electronic signal having an arbitrary-shaped waveform. Parameters of such a signal may depend, at least in part, on its waveshape. For example, if a signal is graphed as voltage versus time, energy per pulse of the signal may be proportional to the area under the graph. Accordingly, energy per pulse may change as a shape of the pulse changes.

In an embodiment, properties of a signal that is subject to proposed changes may be predicted to determine if such changes give rise to a modified signal that violates particular criteria. For example, a physician may use a computer interface (e.g., touchscreen) to graphically widen a pulse of a signal applied to a patient to an extent that energy of the widened pulse would exceed a regulatory limit. In one implementation, after determining that a proposed change would lead to a signal that no longer meets particular criteria, a system may disallow the proposed change. In another implementation, a system may regulate such a proposed change so that the proposed change is implemented gradually until just before the changing signal no longer meets the particular criteria. Such gradual implementation may allow a system to iteratively change a signal by relatively small steps. For example, an iteration may comprise: change the signal by only a portion of the proposed change; determine whether the portionally-changed signal would violate particular criteria; if not, then further change the signal by another portion of the proposed change; determine whether the portionally-changed signal would violate the particular criteria; if not, then further change the signal by yet another portion of the proposed change; and so on. Such an iterative process may be repeated until a determination is made that the portionally-changed signal would violate the particular criteria. The last modified signal that would not violate the particular criteria may be accepted and provided to an output port, for example.

Accordingly, an embodiment of a method may comprise: receiving a request to change a waveform of a first signal by an amount to a proposed signal. Upon or after determining that the proposed signal would violate one or more particular criteria (which may be stored in a memory, for example), the method may further comprise iteratively changing the first signal by incremental portions of the amount until the last-changed first signal would violate the one or more particular criteria. In one implementation, the allowed signal change (e.g., the signal provided to an output port) may comprise the final iteratively-changed first signal just before violation of the criteria.

In another embodiment, a method may comprise receiving a proposal to change a first electrical signal to a second electrical signal. Such a proposal may comprise a wave shape change of a graphical representation of the first electrical signal made by a user interacting with a graphical display (e.g., touchscreen, or using a mouse and display). The method may further comprise predicting one or more parameters of the second electrical signal, and comparing the one or more parameters to corresponding one or more threshold values. Parameters may comprise power output or frequency, for example. Threshold values, for example, may be established by criteria set forth by a regulatory entity. The method may further comprise determining whether to reject or accept the proposal based, at least in part, on comparing the parameters to the corresponding threshold values. In one implementation, a modified second electrical signal may be determined in response to the one or more parameters exceeding any of the corresponding one or more threshold values. For example, if the second signal would violate criteria (e.g., which include the threshold values), then a method may include determining a modified second signal (e.g., a third signal) that would not violate criteria. Of course, details of such a method are merely examples, and claimed subject matter is not so limited.

In some implementations, a proposal to change one signal to another signal may be generated graphically. For example, a user may graphically drag portions of a graphical representation of a signal pulse using a mouse to change the shape of the pulse. In other implementations, a proposal to change a first signal to another signal may be generated in response to feedback based, at least in part, on the first electrical signal applied to a subject. In yet other implementations, a proposal to change a first signal to another signal may be generated in response to a user adjusting a knob or other control device, such as a user increasing an intensity of a signal. Proposed changes of a signal may comprise a proposal to change a shape of the signal, a frequency (or frequencies) of the signal, a magnitude of the signal, and so on.

In another embodiment, a method may comprise: applying a first electrical signal to a biological subject; identifying a proposal to change the first electrical signal to a proposed second electrical signal; calculating at least one parameter of the proposed second electrical signal; and determining whether the calculated at least one parameter violates any rules. If at least one parameter does violate any of the rules, then: iteratively modifying one or more features of the proposed second electrical signal to arrive at a modified second electrical signal that complies with the rules. However, if the at least one parameter does not violate any of the rules, then: applying the proposed second electrical signal to the subject. The method may further comprise generating a proposal in response to feedback based, at least in part, on the first electrical signal applied to the biological subject. In another implementation, the method may further comprise generating a proposal in response to output of one or more detectors based, at least in part, on a physical condition of a biological subject.

In various implementations: such rules may comprise maximum rates of change of the one or more parameters; a proposal may be generated by a user via a mouse or a touchscreen; a proposal may comprise a graphical representation of the proposed second electrical signal; a proposal to change the first electrical signal may comprise a proposal to change a shape of the first electrical signal; and rules may comprise one or more limit values set forth by a government regulatory entity or by a biological subject, for example. Of course, details of such a method are merely examples, and claimed subject matter is not so limited.

In another embodiment, an apparatus may perform methods described above, for example. Such an apparatus may comprise: a display to receive a graphically generated proposal to change a first electrical signal to a second electrical signal, and a processor to: predict one or more parameters of the second electrical signal; and to determine whether the second electrical signal would violate regulatory criteria based, at least in part, on the one or more parameters. The apparatus may further comprise an output port to provide the second electrical signal if the second electrical signal would not violate the regulatory criteria. In one implementation, the graphically generated proposal may comprise a graphical representation of the second electrical signal. Of course, details of such an apparatus are merely examples, and claimed subject matter is not so limited.

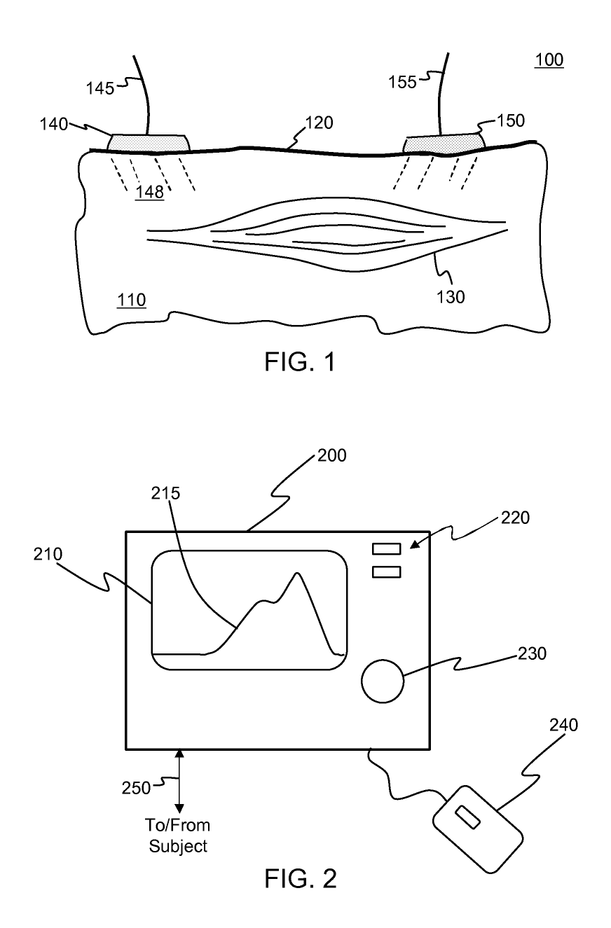

FIG. 1 is a cross-sectional schematic diagram illustrating electrodes 140 and 150 for applying or distributing one or more electrical signals to a portion 110 of a subject, according to an embodiment 100. Portion 110 may comprise a volume of body mass including skin 120 and muscle 130. For sake of clarity, portion 110 may include other biological elements or material that which are not shown. For example, such biological elements or material may comprise DNA, normal or cancer cells, fascia, bone, ligaments, organs, plasma, blood vessels, arteries, and so on. Leads 145 and 155 may carry electrical signals to/from electrodes 140 and 150, respectively. A general flow of electrical signals is schematically indicated by symbol 148. Electrodes 140 and 150 may comprise a self-adhesive, metal foil, or conductive rubber (e.g., carbon-impregnated silicone rubber) electrode. In some implementations, a coupling medium may be used to provide a conductive bridge between the electrode and the skin, such as by filling in voids or gaps, or by increasing conductivity of skin or electrode surfaces. A coupling medium may be an integral part of self-adhesive electrodes, for example. With conductive rubber electrodes an adhesive gel pad may be used. A coupling gel-pad, which may be solid but soft and flexible, may be both electrically conductive and adhesive. Electrodes may also be strapped onto skin, with or without a coupling medium. A coupling medium for metal foil electrodes may comprise an electrode gel or a wetted pad of lint, cotton gauze, or some form of sponge material that absorbs and retains water, for example. Metal electrodes using spread-able gel or wetted pads may be held in contact to skin by straps or bandages.

FIG. 2 is a schematic diagram illustrating at device 200 displaying a graphical representation of a signal 215 that may be applied to a subject (e.g., 1440 in FIG. 14) via port 250, according to an embodiment. Device 200 may generate an electrical signal to be applied to a subject via electrodes, for example, such as 140 and 150. The signal may comprise a wave that may attain any of a number of shapes. For example, the signal may comprise a sinusoid, a square wave, a sawtooth wave, a low-duty-cycle pulse, or an arbitrarily-shaped wave. In the example shown, signal 215 may comprise a pulse including two peaks. Such signals are merely examples, and claimed subject matter is not limited to any particularly-shaped wave or signal. Device 200 may include a screen 210, which may comprise a touchscreen, for example. Device 200 may include a number of switches 220 or knobs 230 to allow a user to manipulate the device. In one implementation, a graphical representation of signal 215 may be changed by a user via touchscreen 210, explained in further detail below. In another implementation, a graphical representation of signal 215 may be changed by a user via mouse 240. In yet another implementation, signal 215 may be changed in response to feedback or other signal provided at port 250, as explained below. Of course, such details of device 200 are merely examples, and claimed subject matter is not so limited.

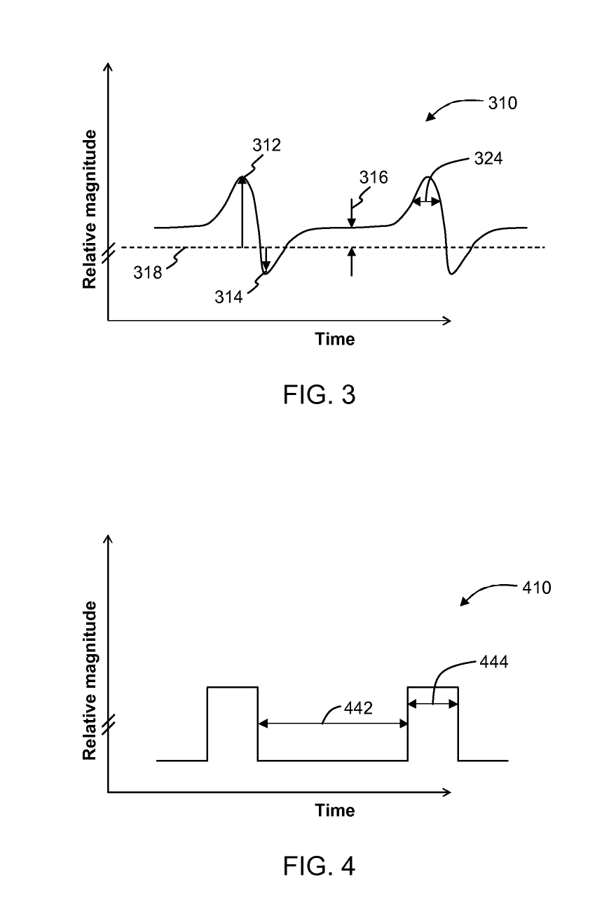

FIGS. 3-6 show example signal waves plotted as magnitude of voltage or current versus time, according to embodiments. For example, a first or second signal applied to a subject via electrodes may comprise any such wave or variation thereof. Of course, there are an endless variety of waves having different shapes or characteristics, and FIGS. 3-6 show merely a small number of possibilities. Here, the figures are useful for helping to explain meanings of some terms that are used to describe signal characteristics.

In particular, FIG. 3 shows a wave 310 that includes a positive-going peak magnitude 312 (e.g., curve is concave downward), a negative-going peak magnitude 314 (e.g., curve is concave upward), and an offset 316 from a reference level 318, which may be zero volts or ground, for example. Wave 310 also includes a width 324 (e.g., pulse width), which may be described as full width at half max (FWHM). In FIG. 4, wave 410 comprises a square wave having a pulse width 444 and duty cycle that may be described by time 442 between pulses. Of course, any wave may be described by any parameters introduces above, and claimed subject matter is not so limited.

In a particular implementation, a waveshape of a first signal may change to arrive at a waveshape of a second signal by any amount or fashion. Such a change may be desired, or proposed, by a user based, at least in part, on a particular situation at hand. For example, a user may desire to widen a pulse of a signal because a wider signal may speed a process of measuring a biological state of a patient. Of course, such situations are merely examples are, and claimed subject matter is not so limited.

In a particular implementation, intensity values of a first signal may change by any amount or fashion. Such a change may be desired, or proposed, by a user based, at least in part, on a particular situation at hand. For example, a user may desire to increase a value of one portion of a signal while decreasing another portion of the signal because such a change may affect a particular organ of a patient over another. Here, the meaning of “intensity values” may include values of voltage or current of any portion of a wave, such as a positive peak (e.g., 312), a negative peak (e.g., 314), an offset (e.g., 316) of a wave from a reference (e.g., ground), and so on.

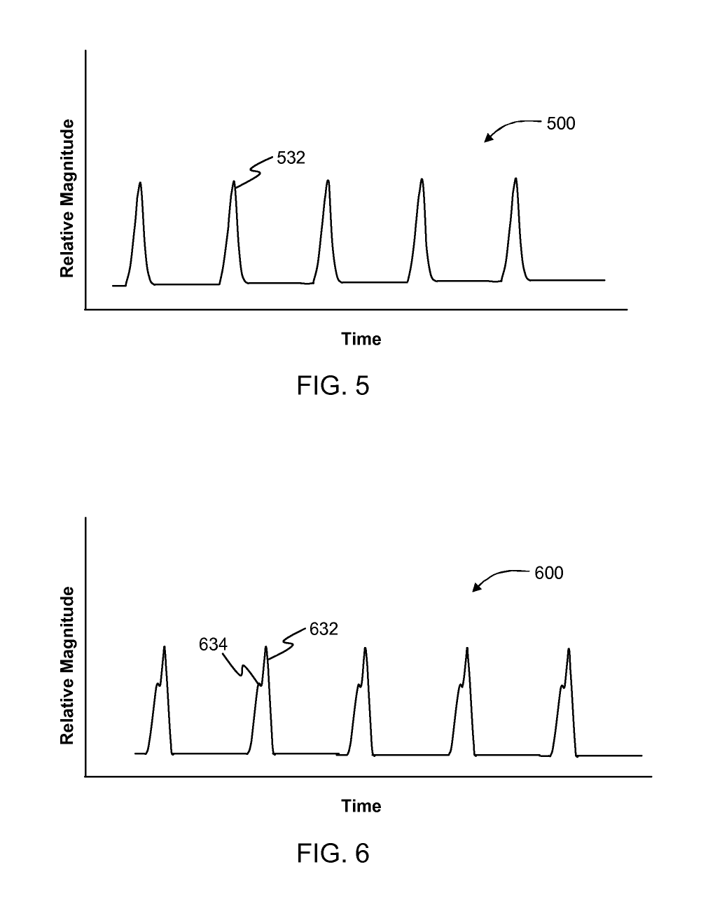

FIG. 5 shows a plot of a signal 500 comprising pulses 532 having a first shape. FIG. 6 shows a plot of a signal 600 comprising pulses 632 having a second shape, including a feature 634. In one implementation, for example, pulse 532 may be graphically manipulated to create feature 634, thus arriving at signal 600.

While signal 500 may not violate any particular criteria, signal 600 might. For example, a regulatory agency may stipulate that energy of a pulse applied to a patient may not exceed 300 milli-joules (mJ). Pulse 532 may have energy of 295 mJ, and thus satisfies this upper energy limit set forth by the agency. However, graphical manipulation of pulse 532 may lead to feature 634, which may push the energy of the resulting pulse 632 to over the 300 mJ limit. For example, the energy of a pulse may be proportional to the area under the pulse curve.

In one implementation, a graphical representation (e.g., magnitude versus time) of a portion of an electric signal may be displayed in a display. For example, an oscilloscope used to measure a signal may perform such a task. In another example, a system to apply an electric signal to a subject may include an output device to indicate to a user any of a number of characteristics of the signal. One example of such an output device may include a touchscreen or LCD display to graph magnitude (e.g., voltage or current) of an electric signal versus time. Another example of such an output device may include a display to list numbers (such as in a table) representative of an electric signal.

In some embodiments, a touchscreen display may function as an output device to display a graphical representation of an electric signal, as mentioned above. A touchscreen display may also function as an input device to receive user instructions to modify a graphical representation of an electric signal shown in the display. From time to time or periodically, a processor may poll a touchscreen display for any such user instructions, though claimed subject matter is not limited to any particular technique of conveying information from a display to a processor. Herein, the meaning of “a processor performing an activity” comprises “a processor executing code to perform an activity”, unless the context of usage in a sentence clearly indicates otherwise.

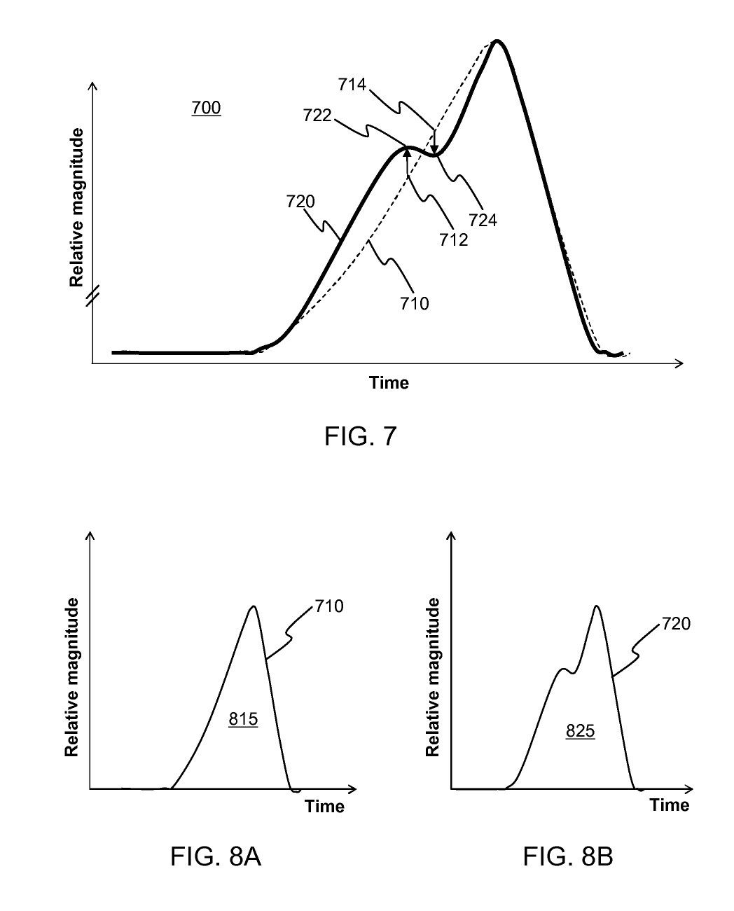

FIG. 7 shows a plot 700 of a first signal 710 modified to a second signal 720, plotted as magnitude of voltage or current versus time, according to an embodiment. For example, plot 700 may be displayed in an LCD, projector display, or touchscreen display. First signal 710 may be applied to a subject in real-time, as a graphical representation of the signal is displayed, for example. First signal may comprise a pulse wave having a frequency of several hundred hertz and a peak voltage of 50 volts, just to give a particular numeric example. Of course, claimed subject matter is not limited to any such values. Frequencies may range from 0 Hz to megahertz, and voltages may range from 0 to hundreds of volts or more and, again, claimed subject matter is not limited in this respect.

In one implementation, a user may (instruct a processor to) modify one or more features of first signal 710 using any of a number of techniques (to communicate to the processor). In one technique, a user may use a mouse to drag portions of the graphical representation of signal 710. For example, a user using a mouse may drag a portion 712 of the graphical representation of 710 upward to point 722 and drag portion 714 of the graphical representation of 710 to point 724. In another example, a user using a finger or stylus on a touchscreen may drag a portion 712 of the graphical representation of 710 upward to point 722 and drag portion 714 of the graphical representation of 710 to point 724. A processor receiving signals from a mouse or touchscreen may redraw a modified curve 720 to represent a graphical representation of second signal 720.

In another technique, a user may use a knob or other control device to adjust some aspect of signal 710 or the graphical representation of signal 710. For example, a user using a knob may desire to increase amplitude of signal 710 to a particular value. A processor receiving signals from the knob may redraw a modified curve 720 to represent a graphical representation of second signal 720, though such a drawing process is optional, and claimed subject matter is not so limited.

Though a user may modify signal 710 to any configuration, such modification may be constrained to comply with various criteria, such as those set forth by a regulatory agency. Accordingly, in one embodiment, second signal 720 may be considered a proposed signal 720, which may be evaluated or tested for compliance with any of a number of such criteria, as described below. A processor may draw 720 on a screen while not actually generating or providing a signal 720 at an output port. If 720 passes (e.g., by not violating any such criteria), then signal 720 may be generated and provided at an output port. If 720 fails tests, then a processor may modify signal 720 so that a modified 720 passes the tests, and subsequently may generate and provide the modified 720 at an output port. In another implementation, an alarm (e.g., audible or a light) may be activated in response to failure of a test.

Any of a number of features of signals 710 or 720 may be evaluated, including any of a combination of peak or average voltage, peak or average current, energy per pulse, energy per cycle, frequency components, zero-offset, pulse width, slope, decay rate, rise time, and so on. Such evaluation may be in view of one or more criteria or rules set forth by any entity, including a government agency (e.g., the Food and Drug Administration (FDA), Federal Communications Commission (FCC), or Federal Aviation Administration (FAA)) or a committee or governing body of a group (a medical group overseeing patient treatment, an ad hoc group drafting guidelines for testing on animals, and so on), by a human subject (e.g., 1440) to which signals (e.g., 710 or 720) may be applied, by a medical practitioner treating a patient, or by a researcher investigating a subject, just to name a few examples. In one particular implementation, one or more criteria or rules may be based, at least in part, on safety or medical history of a patient, just to name a few examples. For example, a rule may set forth a relatively low maximum voltage of a signal for a patient with a preexisting heart condition. Another rule may set forth a relatively high maximum voltage of a signal for a young, healthy patient. One or more criteria or rules may be stored in a memory. Accordingly, in view of an example modification shown in FIG. 7, signal 710 may pass one or more criteria whereas 720 may fail, or vise versa.

For example, the International Electrotechnical Commission (IEC) sets forth a rule that pulse energy for pulse durations of less than 0.1 seconds shall not exceed 300 mJ per pulse (e.g., IEC 60601-2-10, section 201.12.4.104, Limitation of Output Parameters). As an example, signal 710 may have pulse energy just below 300 mJ. A new signal 720, formed by a user dragging points 712 and 714, may exceed 300 mJ. FIG. 8A shows a pulse of signal 710 having an area 815. FIG. 8B shows a pulse of signal 720 having an area 825. In these examples, it can be seen that area of a pulse may increase by modifying 710 by moving points (e.g., 712, 714). Pulse energy of a signal may be proportional to area under a curve representing the signal.

In another example, a human subject to which signals are applied may set forth maximum values of voltage or energy of the signals. In an example implementation, an electrical muscle stimulator may apply an electrical signal transcutaneously to a patient. Though the signal may benefit the patient (and the patient selects such treatment), the signal may be uncomfortable, more so for some patients. Comfort level may be proportional to voltage level of an applied signal, for example. Accordingly, it may be desirable for patients to have an opportunity to select maximum values of voltage or energy, among other things, of the signals based, at least in part, on the patient's anticipated comfort level, for example.

In an example embodiment, a method may comprise receiving a proposal to change a first signal 710 to a proposed second signal 720. Such a proposal may be generated at a user interface, such as a touchscreen, touchpad, screen with mouse interaction, and so on. Or such a proposal may be generated by a knob or other control device that a user may operate. A processor (e.g., processing unit 1520) receiving the proposal may predict one or more parameters of the second signal, and may compare the one or more parameters to criteria or corresponding one or more threshold values. For example, such parameters may comprise any of a number of electrical characteristics of the proposed second signal, such as energy per pulse, peak voltage, current, and so on. In one implementation, a processor may compare one or more parameters of the proposed second signal with those of the first signal. For example, there may be criteria that specify by how much certain parameters of a first signal may change to arrive at a second signal. For instance, one rule set forth by an agency may specify that peak voltage of a pulse applied to a medical patient may not incrementally increase by more than 5% of the original value. The processor may subsequently determine whether to reject or accept the proposed second signal based, at least in part, on the comparing of parameters to threshold values or whether the second signal complies with the criteria.

In one implementation, a processor may determine a modified second signal in response to one or more parameters exceeding any corresponding one or more threshold values. For example, if a processor determines that proposed second signal 720 involves an energy per pulse that exceeds a threshold set forth by an agency, then the processor may modify the proposed second signal 720 to a third signal that has a reduced energy per pulse. The processor may generate such a third signal so that the third signal does not exceed this threshold. In one implementation, the processor may propose the third signal to a user for user's approval before generating or providing the third signal to an output port. In another implementation, the processor may forego a user-approval step and immediately generate or provide the third signal to an output port, and maybe to a display.

In some embodiments, a proposal to change a first signal to a second signal may be generated by a knob or other control device that a user may operate. For example, a user may desire to increase amplitude of a signal to a particular value. However, such a particular value, in view of other parameters (e.g., any of a combination of peak or average voltage, peak or average current, energy per pulse, energy per cycle, frequency components, zero-offset, pulse width, slope, decay rate, rise time, and so on) may lead to a second signal that would violate criteria or rules. For example, a relatively narrow pulse width may allow for a relatively high peak voltage, whereas a relatively wide pulse width may preclude such a relatively high peak voltage. Accordingly, a processor receiving the proposal may predict one or more parameters of the second signal, and may compare the one or more parameters to criteria such as corresponding one or more threshold values. The processor may subsequently determine whether to reject or accept the proposed second signal based, at least in part, on the comparing of parameters to threshold values or whether the second signal complies with the criteria.

In some embodiments, a proposal to change a first signal to a second signal may be generated graphically (e.g., by a user via a mouse or touchscreen), as described above. In other embodiments, a proposal to change a first signal to a second signal may be generated in response to feedback based, at least in part, on the first signal applied to a subject. For example, a first signal may be applied transcutaneously via cables and electrical pads to a patient. A response of a subject (e.g., patient) to a first signal may give rise to a feedback signal that may be representative of a physical condition of the subject, as described below. In still other embodiments, a proposal to change a first signal to a second signal may be generated by a knob or other control device that a user may operate. For example, a user may desire to increase amplitude of a signal to a particular value.

As indicated above, a first signal may be used as a diagnostic tool to measure impedance of biological elements of the patient: An electrical signal may follow a path depending, at least in part, on electrical and/or chemical properties of internal portions of a subject. For example, electrical conductivity of muscle may be different from that of bone or a particular organ. Moreover, as an example, electrical conductivity of muscle tissue or bone may depend, at least in part, on the health or density of the muscle tissue or bone (or portion thereof). In the case of muscle tissue, for example, measurements of electrical conductivity of muscle tissue may be used to determine muscle loss or gain in subjects with Lou Gehrig's Disease, also known as amyotrophic lateral sclerosis, or ALS. This disease may attack motor neurons that control voluntary muscle movement, leading to muscle weakness and atrophy. As ALS spreads, motor neurons may die off, causing muscles to atrophy. Deteriorating muscles may behave differently from healthy ones, resisting electrical current more, for example. Such variations in behavior may be correlated with disease progression and length of survival of a subject. As another example, electrical conductivity of internal portions of a subject may depend, at least in part, on tissue density, presence of cancer cells, and so on.

Biological elements may respond to different signals in different ways. For example, a pulse of a signal may activate an action potential of nerve fibers in muscle tissue if a slope of the pulse is sufficiently steep. On the other hand, if a pulse is not steep enough, then the same nerve fibers may accommodate (e.g., “adjust”) to current flow of the pulse so that no action potential is activated. This illustrates an example where applied signals may affect biological elements for which the signals are used to diagnose. For another such example, a signal applied to muscle tissue may increase permeability of the muscle tissue. Accordingly, application of particular signals may affect muscle tissue so that resistance of the muscle tissue changes in response to the applied signals. Different applied signals (e.g., different by frequency, waveshape, voltage level, and so on) may affect particular biological elements differently. Thus, for example, different applied signals may give rise to different resistances of a particular biological element, which may give rise to particular feedback signals feedback to the source of the applied signals.

A feedback signal may travel in the same cables and electrical pads as that of the first signal or may travel in a second set of cables and electrical pads. In response, at least in part, to evaluating a feedback signal, a processor may execute code that determines whether or not the first signal is to be changed, and if so, what updates or changes are to be made to arrive at a second signal to apply to the subject. For example, a processor may determine that a feedback signal based, at least in part, on a first signal applied to a patient indicates that a voltage of the first signal should be increased by a particular amount to have a desired affect on the patient. The processor may consequently generate a second signal having an increased voltage. However, the processor may further determine whether such a second signal (changed from a first signal) would violate any rules or criteria. For example, an increased voltage of a second signal may not violate any criteria, but an associated increase in power may violate criteria. A processor may evaluate a number of parameters to arrive at a second signal that finally satisfies a feedback signal and applicable criteria, or may place uneven weight on the feedback signal, the criteria, and/or signal parameters to reach a compromise. Other changes to a signal, besides voltage, may involve changing its shape, frequencies, magnitude, power, and so on. The second voltage may be proposed to a user for approval or may forego user approval and be provided to an output port (and to the patient). Of course, such details of processes involving feedback are merely examples, and claimed subject matter is not so limited.

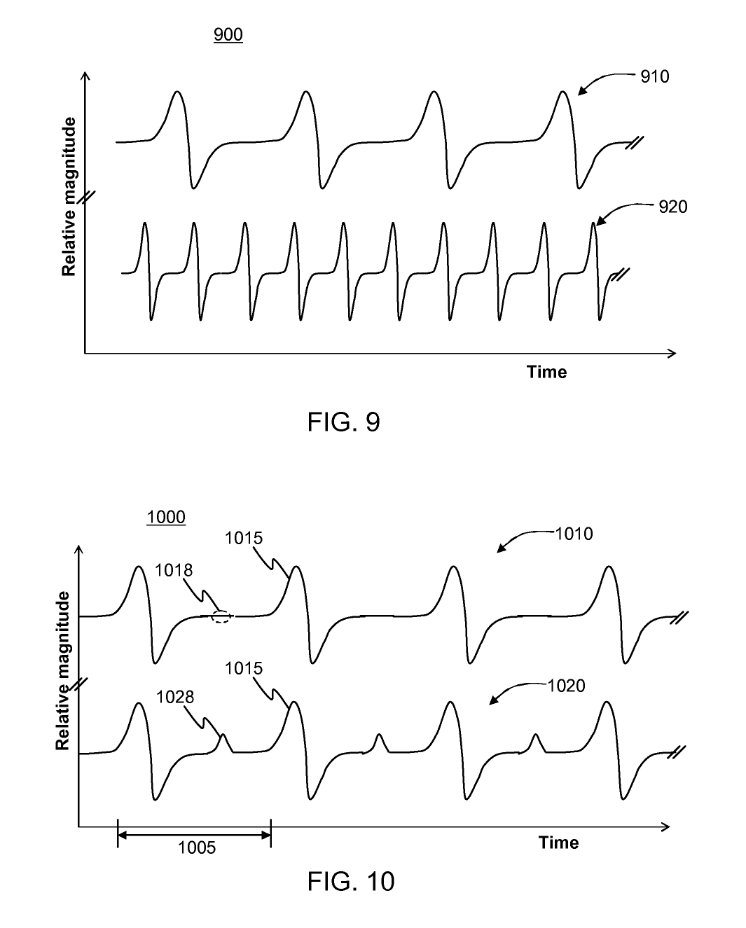

FIG. 9 shows a graphical representation of a first signal 910 and a graphical representation of a second signal 920 plotted as magnitude of voltage or current versus time, according to embodiments 900. First signal 910 and second signal 920 have different frequencies. Second signal 920 may be derived from first signal 910 by a user numerically changing the frequency of the first signal (e.g., user modification of a signal need not be performed via graphical interaction).

FIG. 10 shows a graphical representation of a first signal 1010 and a graphical representation of a second signal 1020 plotted as magnitude of voltage or current versus time, according to embodiments 1000. In this example embodiment, a user may have graphically modified a graphical representation of first signal 1010 by dragging a portion 1018 of the graphical representation of the signal to form an inter-pulse peak 1028 between pulses 1015. In this example embodiment, such a modification may involve an increase in energy per cycle, an increase in average voltage or current, among other changes. Such a modification may also involve a change in Fourier frequencies: Signal 1010 may have one particular distribution of Fourier frequencies, and signal 1020 may have another particular distribution of Fourier frequencies. For example, by adding the feature 1028, a new Fourier distribution of frequencies may be created. In the example of embodiment 1000, a Fourier distribution of frequencies of signal 1020 may include frequency components that are about double that of signal 1010, since about double the number of pulses per cycle 1005 may arise due to peak 1028.

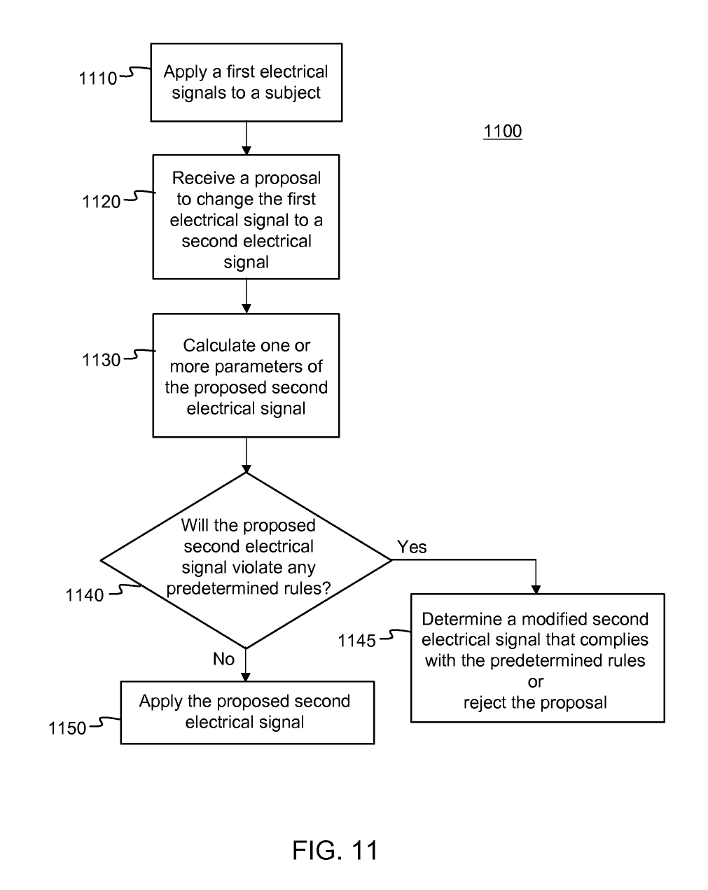

FIG. 11 is a flow diagram of a process 1100 for determining whether to modify a signal, according to an embodiment. At block 1110, a first signal may be applied to a subject, such as a patient. At block 1120, a processor may receive or identify a proposal to change the first signal to a proposed second signal. Such a proposal may be initiated by a user graphically modifying a graphical representation of the first signal. Or such a proposal may be initiated by a processor in response, at least in part, to feedback based, at least in part, on the first signal, as described above. In another implementation, such a proposal may be initiated by a processor in response, at least in part, to a signal from a source such as a detector, transducer, and so on. For example, a detector may measure a skin condition (e.g., pH value, moisture content, etc.), heart rate, blood pressure, blood oxygen, or other physical condition of a patient. A proposal to change a first signal to a proposed second signal may be based, at least in part, on output from one or more of such type of detectors.

At block 1130, the processor may calculate or evaluate one or more parameters of the proposed second signal, such as peak voltage, energy per cycle, or frequency, just to name a few examples. At diamond 1140, a determination may be made as to whether the proposed second signal having the calculated parameters would violate any rules. In other words, the processor may determine whether a signal having particular values of parameters set forth by the proposed second signal would violate any rules. If so, then process 1100 may proceed to block 1145 where a processor may iteratively modify one or more features of the proposed second signal to arrive at a modified second signal that does not violate the rules. In one alternate implementation, the proposal may be rejected and the user may take another course of action, such as initiating another proposal. In yet another alternate implementation, the proposal may be rejected and an audible or visual alarm may be activated. On the other hand, if the proposed signal does not violate any rules, then process 1100 may proceed to block 1150, where the proposed second signal may be applied to the subject. Of course, such details of process 1100 are merely examples, and claimed subject matter is not so limited.

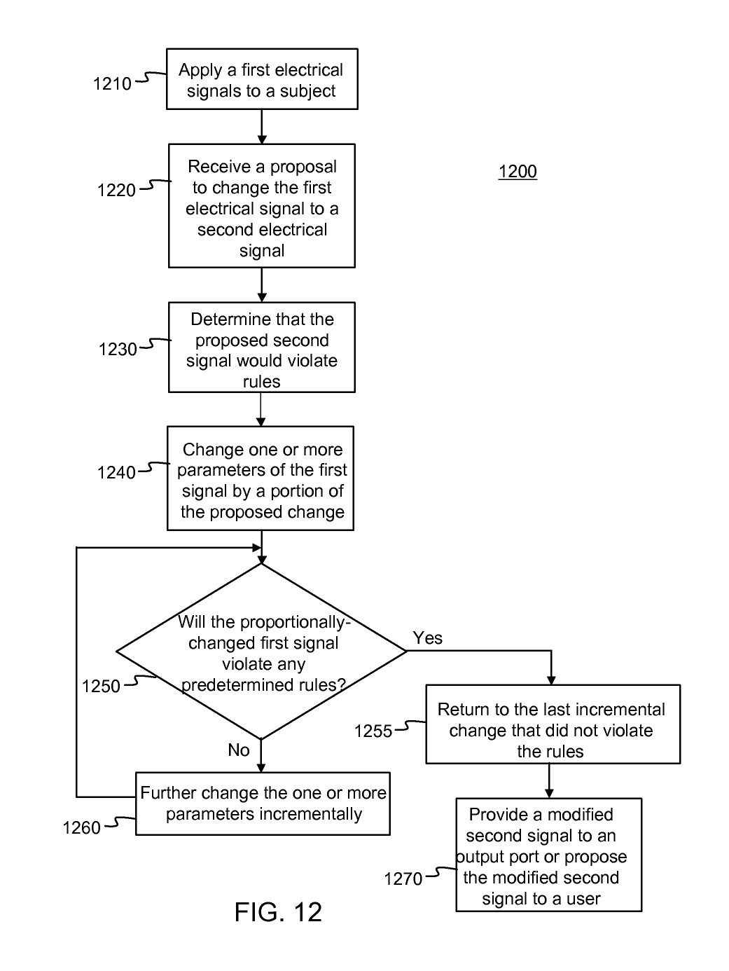

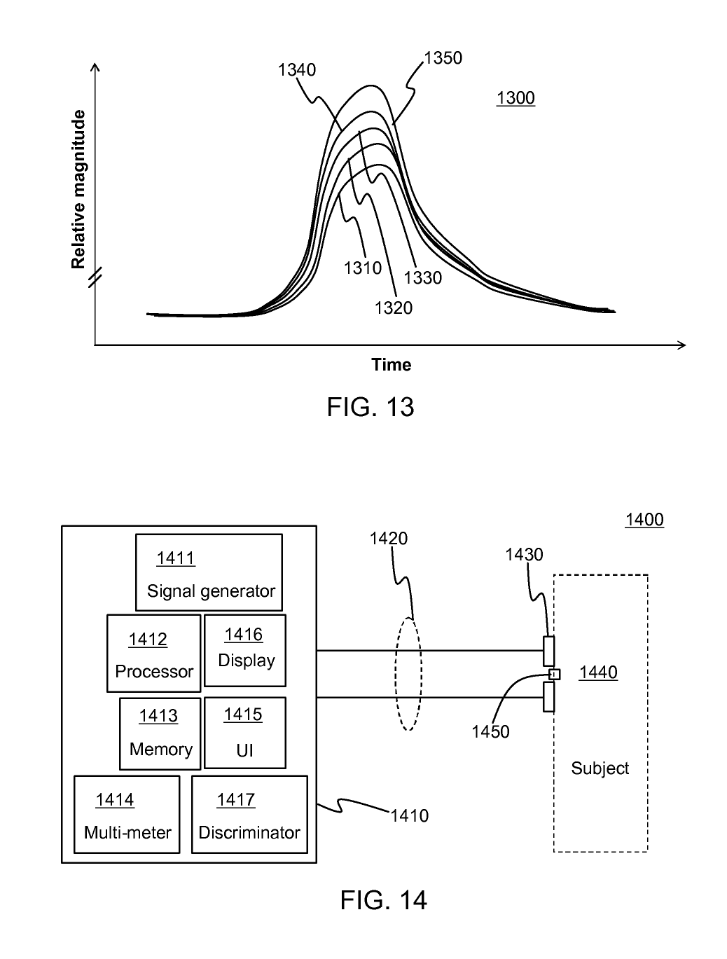

FIG. 12 is a flow diagram of a process 1200 for modifying a proposed signal, according to another embodiment. FIG. 13 shows a first signal wave 1310 in a process of being modified to a second signal wave, plotted as magnitude of voltage or current versus time, according to an embodiment 1300. At block 1210, first signal 1310 may be applied to a subject, such as a patient. At block 1220, a processor may receive or identify a proposal to change first signal 1310 to a proposed second signal 1350. Such a proposal may be initiated by a user graphically modifying a graphical representation of the first signal. Or such a proposal may be initiated by a processor in response, at least in part, to feedback based, at least in part, on the first signal, as described above. Or such a proposal may be initiated by particular output values of one or more detectors measuring parameters representative of a physical condition of a subject, as described above.

At block 1230, a determination may be made that the proposed second signal 1350 would violate rules, such as those set forth by an agency or group, for example. Such a determination may be made by a processor calculating or evaluating one or more parameters of the proposed second signal, such as peak voltage, energy per cycle, frequency, just to name a few examples. In other words, a processor may determine that a signal having particular values of parameters set forth by the proposed second signal would violate at least one rule.

At block 1240, a processor may change one or more parameters of first signal 1310 by relatively small incremental amounts, so that a resulting signal 1320 is modified by merely a portion of the proposed modification. The intent is that first signal 1310 is to be modified gradually to determine at which modification stage rule violation occurs. At diamond 1250, a determination may be made as to whether the gradually modified signal 1320 violates any rules. If not, then process 1200 may proceed to block 1260, where signal 1320 may be further modified by incremental changes of one or more parameters to arrive at signal 1330. Process 1200 may then return to diamond 1250, where a determination may be made as to whether the gradually modified signal 1330 violates any rules. If not, then process 1200 may proceed to block 1260, where signal 1320 may again be further modified by incremental changes of one or more parameters to arrive at signal 1340. Process 1200 may then return to diamond 1250, where a determination may again be made as to whether the gradually modified signal 1340 violates any rules. If not, then process 1200 may proceed to block 1260, and this portion of process may repeat. However, if the gradually modified signal 1340 violates any rules, then process 1200 may proceed to block 1255, to return to parameter values of the most recent signal 1330 that did not violate any rules. At block 1270, signal 1330 may be provided to an output port or proposed as a modified second signal to a user, for example. Of course, such details of process 1200 are merely examples, and claimed subject matter is not so limited.

In another embodiment, portions of process 1200 may be reversed. For example, process 1200 comprises blocks 1240, 1255, 1260, 1270, and diamond 1250, which set forth changing one or more parameters of a first signal by relatively small incremental amounts so that a resulting signal is modified by merely a portion of the proposed modification; determining whether the gradually modified signal violates any rules; if not, then further modify the signal by incremental changes of one or more parameters; repeat until the gradually modified signal violates rules; then return to parameter values of the most recent signal that did not violate any rules. The resulting modified signal may then be provided to an output port or proposed as a modified second signal to a user, for example. A reversed process may set forth changing one or more parameters of a proposed second signal by relatively small incremental amounts; determining whether the gradually modified signal still violates any rules; if so, then further modify the signal by incremental changes of one or more parameters; repeat until the gradually modified signal no longer violates rules; the resulting modified signal may then be provided to an output port or proposed as a modified second signal to a user, for example. Of course, such details of this alternative process are merely examples, and claimed subject matter is not so limited.

FIG. 14 is a schematic block diagram illustrating a system 1400 for performing a process, such as 1100 or 1200, for example, according to an embodiment. For example, system 1400 may comprise a device 1410, cables 1420, and electrodes 1430. Device 1410 may generate one or more signals that may be applied to a subject 1440 via electrodes 1430. Device 1410 may include a signal generator 1411 to generate signals having any of a number of parameters, such as waveshape, magnitude, frequency, offset (e.g., from zero volts), and so on. Signal generator 1411 may generate more than one signal at a time, or may repeatedly and alternately generate a first signal and a second signal. A processor 1412 may be used to calculate or determine resistance to a signal provided to electrodes 1430, which may be electrically connected to subject 1440. Processor 1412 may also evaluate feedback provided by cables 1420 to determine any of a number of parameters. In another implementation, processor 1412 may also evaluate output of detectors 1450 provided via cables 1420, other conductors, or wireless transmission (e.g., from detector 1450 to device 1410). Such detectors may measure one or more parameters representative of a physical condition of subject 1440. For example, such detectors may comprise a blood pressure monitor, blood oxygen level monitor, and so on. Processor 1412 may perform evaluations, calculations, or determinations using parameters measured by multi-meter 1414, for example. Such parameters may include voltage, current, phase shift, and so on.

A discriminator 1417 may decompose or separate a composite (e.g., non-sinusoidal) signal into two or more individual signals. In one implementation, a composite voltage signal may include a superposition of any number of individual voltage signals. Current of the composite voltage signal flowing through subject 1440 may be decomposed by discriminator 1417 so that the current is separated into a number of individual current signals, which may be measured by multi-meter 1414, for example. In one implementation, discriminator 1417 may comprise one or more frequency filters (e.g., low-pass, high-pass, or notch filters, and so on) to perform such signal separation. In another implementation, discriminator 1417 may comprise one or more amplitude filters (e.g., involving resistor networks, diodes, etc.) to perform such signal separation. In yet another implementation, discriminator 1417 may comprise one or more waveshape filters to perform such signal separation. In any case, a composite signal provided to discriminator 1417 (e.g., by cables 1420) may comprise a digital signal. Here, an analog to digital converter (not shown) may be used to convert an analog composite signal flowing through subject 1440 to a digital composite signal. Software executed by processor 1412 may be used to identify or distinguish one waveform of one signal from another waveform of another signal in a digital composite signal. With information from such a processor, discriminator 1417 may separate the separate waveforms and multi-meter 1414 may then measure current or voltage of the separated waveforms.

Device 1410 may further include memory 1413 to store values of parameters measured by multi-meter 1414, or generated by processor 1412 or discriminator 1417, for example. Memory 1413 may also maintain data representative of criteria, rules, or regulations set forth by an agency, group, and so on. Memory 1413 may also store values produced by detectors 1450, for example. Data may comprise tables of values of ranges, maxima, minima, averages, etc. for any of a number of parameters of a signal, such as voltage, current, energy, power, rate of change, and so on. A user interface 1415 may include a keypad, mouse, or touchscreen by which a user may provide operational instructions to device 1410. A display 1416 may display any information to a user, including a graphical representation of a signal provided over cables 1420, or a proposed signal. Display 1416 may comprise a portion of user interface 1415, and may comprise a touchscreen, touchpad, and so on. Graphical data in display 1416 may be read by processor 1412 in a process of transferring a graphical representation of a signal from display 1416 to digital values stored in memory 1413. Display 1416 may display a graphical representation of a signal that is present on cables 1420 or may display a graphical representation of a virtual signal that is merely proposed so as to not actually be present on cables 1420. Of course, such details of system 1400 are merely examples, and claimed subject matter is not so limited.

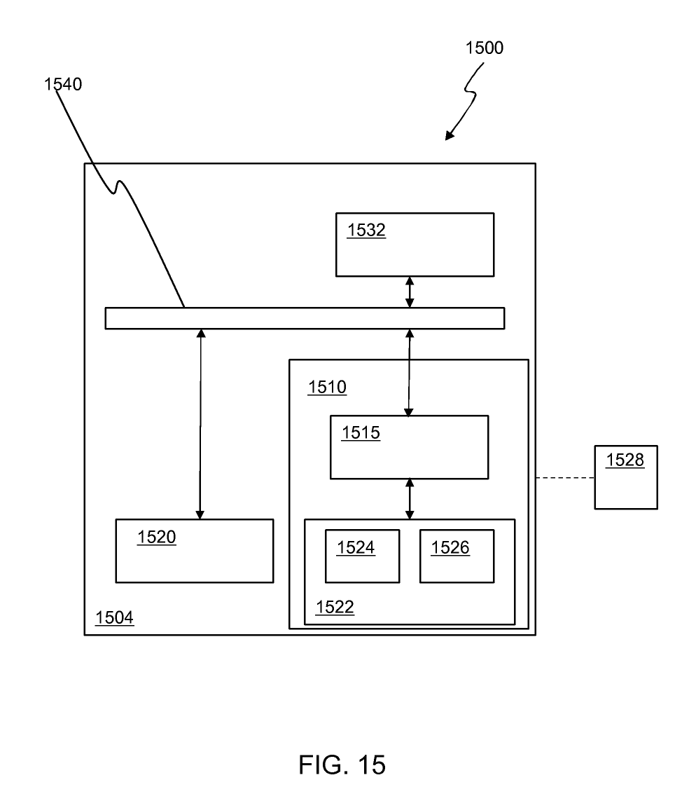

FIG. 15 is a schematic diagram illustrating an embodiment of a computing system 1500, for example. Some portions of system 1500 may overlap with some portions of system 1400. System 1500 may be used to perform processes 1100 or 1200, for example. A computing device may comprise one or more processors, for example, to execute an application or other code. A computing device 1504 may be representative of any device, appliance, or machine that may be used to manage memory module 1510. Memory module 1510 may include a memory controller 1515 and a memory 1522. By way of example but not limitation, computing device 1504 may include: one or more computing devices or platforms, such as, e.g., a desktop computer, a laptop computer, a workstation, a server device, or the like; one or more personal computing or communication devices or appliances, such as, e.g., a personal digital assistant, mobile communication device, or the like; a computing system or associated service provider capability, such as, e.g., a database or information storage service provider or system; or any combination thereof.

It is recognized that all or part of the various devices shown in system 1500, and the processes and methods as further described herein, may be implemented using or otherwise including at least one of hardware, firmware, or software, other than software by itself. Thus, by way of example, but not limitation, computing device 1504 may include at least one processing unit 1520 that is operatively coupled to memory 1522 through a bus 1540 and a host or memory controller 1515. Processing unit 1520 is representative of one or more devices capable of performing at least a portion of a computing procedure or process, such as processes 1100 or 1200, for example. By way of example, but not limitation, processing unit 1520 may include one or more processors, microprocessors, controllers, application specific integrated circuits, digital signal processors, programmable logic devices, field programmable gate arrays, and the like, or any combination thereof. Processing unit 1520 may include an operating system to be executed that is capable of communication with memory controller 1515.

In one implementation, an apparatus may comprise a display (e.g., 1532) to receive a graphically generated proposal to change a first electrical signal to a second electrical signal, and a processing unit 1520 to predict one or more parameters of the second electrical signal, and to determine whether the second electrical signal would violate regulatory criteria based, at least in part, on the one or more parameters. The apparatus may further comprise an output port (e.g., 1532) to provide the second electrical signal if the second electrical signal would not violate the regulatory criteria.

An operating system may, for example, generate commands to be sent to memory controller 1515 over or via bus 1540. Commands may comprise read or write commands, for example. In response to a write command, for example, memory controller 1515 may perform process 1500 described above, to program memory and to change parity states.

Memory 1522 is representative of any information storage mechanism. Memory may store rules or criteria, signals applied to a subject, output from detectors measuring parameters of a subject, an so on, as explained above. Memory 1522 may include, for example, a primary memory 1524 or a secondary memory 1526. Primary memory 1524 may include, for example, a random access memory, read only memory, etc. While illustrated in this example as being separate from processing unit 1520, it should be understood that all or part of primary memory 1524 may be provided within or otherwise co-located or coupled with processing unit 1520. In one implementation, memory 1522 may be incorporated in an integrated circuit, for example, which may comprise a port to receive error syndromes or other ECC information from processing unit 1520.

Secondary memory 1526 may include, for example, the same or similar type of memory as primary memory or one or more other types of information storage devices or systems, such as a disk drive, an optical disc drive, a tape drive, a solid state memory drive, etc. In certain implementations, secondary memory 1526 may be operatively receptive of, or otherwise capable of being operatively coupled to a computer-readable medium 1528. Computer-readable medium 1528 may include, for example, any medium that is able to store, carry, or make accessible readable, writable, or rewritable information, code, or instructions for one or more of device in system 1500. Computing device 1504 may include, for example, an input/output device or unit 1532.

Input/output unit or device 1532 is representative of one or more devices or features that may be capable of accepting or otherwise receiving signal inputs from a human or a machine, or one or more devices or features that may be capable of delivering or otherwise providing signal outputs to be received by a human or a machine. By way of example but not limitation, input/output device 1532 may include a display, speaker, keyboard, mouse, trackball, touchscreen, etc.

It will, of course, be understood that, although particular embodiments have just been described, claimed subject matter is not limited in scope to a particular embodiment or implementation. For example, one embodiment may be in hardware, such as implemented on a device or combination of devices, for example. Likewise, although claimed subject matter is not limited in scope in this respect, one embodiment may comprise one or more articles, such as a storage medium or storage media that may have stored thereon instructions capable of being executed by a specific or special purpose system or apparatus, for example, to lead to performance of an embodiment of a method in accordance with claimed subject matter, such as one of the embodiments previously described, for example. However, claimed subject matter is, of course, not limited to one of the embodiments described necessarily. Furthermore, a specific or special purpose computing platform may include one or more processing units or processors, one or more input/output devices, such as a display, a keyboard or a mouse, or one or more memories, such as static random access memory, dynamic random access memory, flash memory, or a hard drive, although, again, claimed subject matter is not limited in scope to this example.

The terms, “and” and “or” as used herein may include a variety of meanings that will depend at least in part upon the context in which it is used. Typically, “or” if used to associate a list, such as A, B or C, is intended to mean A, B, and C, here used in the inclusive sense, as well as A, B or C, here used in the exclusive sense. Occasionally, the term “and/or” is also used to associate a list in an inclusive and exclusive sense.

Embodiments described herein may include machines, devices, engines, or apparatuses that operate using digital signals. Such signals may comprise electronic signals, optical signals, electromagnetic signals, or any form of energy that provides information between locations.

In the preceding description, various aspects of claimed subject matter have been described. For purposes of explanation, specific numbers, systems, or configurations may have been set forth to provide a thorough understanding of claimed subject matter. However, it should be apparent to one skilled in the art having the benefit of this disclosure that claimed subject matter may be practiced without those specific details. In other instances, features that would be understood by one of ordinary skill were omitted or simplified so as not to obscure claimed subject matter.

While there has been illustrated and described what are presently considered to be example embodiments, it will be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter not be limited to the particular embodiments disclosed, but that such claimed subject matter may also include all embodiments falling within the scope of the appended claims, and equivalents thereof.

What is claimed is:

1. A method comprising:
receiving a graphically generated proposal to change a first electrical signal to a second electrical signal;
predicting one or more parameters of said second electrical signal;
determining whether said second electrical signal would violate regulatory criteria based, at least in part, on said one or more parameters; and
providing said second electrical signal at an output port if said second electrical signal would not violate said regulatory criteria.

2. The method of claim 1, further comprising:
determining a modified second electrical signal that would not violate said regulatory criteria if said second electrical signal would violate said regulatory criteria.

3. The method of claim 1, wherein said regulatory criteria comprise maximum rates of change of said one or more parameters.

4. The method of claim 1, wherein said graphically generated proposal is generated by a user via a mouse or a touchscreen.

5. The method of claim 1, wherein said graphically generated proposal comprises a graphical representation of said second electrical signal.

6. The method of claim 1, wherein said proposal to change said first electrical signal comprises a proposal to change a shape of said first electrical signal.

7. The method of claim 1, wherein said proposal to change said first electrical signal comprises a proposal to change one or more frequencies of said first electrical signal.

8. The method of claim 1, wherein said proposal to change said first electrical signal comprises a proposal to change a magnitude of at least a portion of said first electrical signal.

9. The method of claim 1, wherein said one or more parameters comprise energy, power, or frequency.

10. The method of claim 1, wherein said regulatory criteria comprise one or more limit values set forth by a government regulatory entity.

11. A method comprising:
applying a first electrical signal to a biological subject;
identifying a proposal to change said first electrical signal to a proposed second electrical signal;
calculating at least one parameter of said proposed second electrical signal;
determining whether said calculated at least one parameter violates any rules;
if said at least one parameter does violate any of said rules, then:
iteratively modifying one or more features of said proposed second electrical signal to arrive at a modified second electrical signal that complies with said rules; and
if said at least one parameter does not violate any of said rules, then:
applying said proposed second electrical signal to said subject.

12. The method of claim 11, further comprising:
generating said proposal in response to feedback based, at least in part, on said first electrical signal applied to said biological subject.

13. The method of claim 11, further comprising:
generating said proposal in response to output of one or more detectors based, at least in part, on a physical condition of said biological subject.

14. The method of claim 11, wherein said proposal is generated by a user via a mouse or a touchscreen.

15. The method of claim 11, wherein said proposal comprises a graphical representation of said proposed second electrical signal.

16. The method of claim 11, wherein said proposal to change said first electrical signal comprises a proposal to change a shape of said first electrical signal.

17. The method of claim 11, wherein said rules comprise one or more limit values set forth by a government regulatory entity.

18. The method of claim 11, wherein said rules are based, at least in part, on a medical history of said biological subject.

19. An apparatus comprising:
a display to receive a graphically generated proposal to change a first electrical signal to a second electrical signal;
a processor to:
predict one or more parameters of said second electrical signal; and
determine whether said second electrical signal would violate regulatory criteria based, at least in part, on said one or more parameters; and
an output port to provide said second electrical signal if said second electrical signal would not violate said regulatory criteria.

20. The apparatus of claim 19, wherein said graphically generated proposal comprises a graphical representation of said second electrical signal.
